(19) United States
(12) Patent Application Publication
(10) Pub. No.: US 2013/0331345 A1
(43) Pub. Date: Dec. 12, 2013

(54) PUERARIN HYDRATES, PREPARATION METHODS AND USES THEREOF

(71) Applicant: Li Liu, Foshan (CN)

(72) Inventor: Li Liu, Foshan (CN)

(21) Appl. No.: 13/993,837

(22) PCT Filed: Dec. 17, 2010
(86) PCT No.: PCT/CN2010/002077

§ 371 Date: Aug. 29, 2013

(51) Int. Cl. A61K 31/7048

(52) U.S. Cl. CPC A61K 31/7048
USPC 514/23; 536/1.11; 536/128

(57) ABSTRACT

The present invention pertains to the field of pharmaceutical and chemical engineering, and relates to a puerarin hydrate, the preparation method and use thereof. Specifically, said puerarin hydrate has a molecular formula of C₂₁H₂₀O₉.n H₂O, in which n is a value of 0.8-1.3. The present invention further relates to a pharmaceutical composition comprising said puerarin hydrate, and a method for the treatment of cardiovascular diseases or eye diseases. The puerarin hydrate of the present invention is more stable than puerarin without water of crystallization, convenient for storage and transportation, and has good fluidity at room temperature thereby easy for the manufacture of preparations.

### TECHNICAL FIELD

The present invention pertains to the field of pharmaceutical and chemical engineering, and relates to a puerarin hydrate, the preparation method and use thereof. The present invention further relates to a pharmaceutical composition comprising said puerarin hydrate, and a method for the treatment of cardiovascular diseases or eye diseases.

### BACKGROUND ART

Puerarin has comprehensive pharmacological effects and clinical applications (Research on the pharmacological effects and clinical applications of puerarin, Chinese Journal of Hemorheology, 2004, 14(1):138), but it is reported in references that when puerarin [8-β-D-glucopyranosyl-4′,7-dihydroxylisoflavone, molecular formula: C₂₁H₂₀O₉, molecular weight: 416.37, CAS: 3681-99-0] was administrated via intravenous injection to patients with type II diabetes, it was found that puerarin facilitated improving transudation of thrombus derived trace albuminuria, delaying or preventing occurrence and development of diabetic renal microangiopathy. YU Jian et al observed 48 type II diabetic patients as treated with puerarin, found puerarin had significant functions of reducing blood sugar and improving insulin resistance, and held the opinion that puerarin injection not only was an ideal drug for treatment of insulin resistance of type II diabetic patients, but also had good therapeutic effects on diabetic peripheral neuropathy. On the one hand, puerarin could promote biological activity of insulin, i.e., improving insulin sensitivity via expanding blood vessel, improving microcirculation, increasing blood flow, enhancing ability of blood circulation; on the other hand, puerarin could effectively reducing whole blood viscosity thereby enhancing red cell deformability, increasing red cell membrane elasticity so as to improve its structure and physical properties, and accelerating material transportation and transmembrane ability of glucose and insulin, further improving insulin sensitivity to achieve hypoglycemic effect in type II diabetic patients.

However, puerarin without water of crystallization has a high hygroscopicity, and is prone to deliquescence, so that isolation of air is required to prevent adhesion during processing, and the lack of good sliding property would result in sticking during the manufacture of preparations (e.g., granules or tablets).

So far, there no report about puerarin hydrates (crystalline puerarin hydrates) [C₂₁H₂₀O₉.n H₂O, n is selected from a value ranging from 0.4 to 1.3].

### CONTENTS OF THE INVENTION

The inventors surprisingly find via a lot of experiments and unremitting efforts that puerarin with water of crystallization has a far lower hygroscopicity than that of puerarin without water of crystallization, and is more stable than puerarin without water of crystallization thereby facilitating storage and transportation, and has better fluidity room temperature thereby easy for manufacturing preparations (e.g., granules or tablets). Thus, the following invention is provided.

One aspect of the present invention relates to a puerarin hydrate (crystalline puerarin hydrate), of which the molecular formula is C₂₁H₂₀O₉.n H₂O, wherein, n is a value selected from 0.4-1.3 or 0.8-1.3, for example, n is 0.5, 0.8, 0.85, 0.95, 1, 1.05, 1.1, 1.15, 1.2, 1.25 or 1.3.

Another aspect of the present invention relates to a method for preparing the puerarin hydrate aforesaid, which is any one of the following methods A to E.

Method A:

In a reaction container, subjecting Radix Puerariae powder to cold extraction, ultrasonic extraction, microwave extraction or refluxing extraction one or more times with one or more of water or C1-C6 lower alcohols, combining, filtering, filtering with microporous membrane or ceramic membrane, concentrating filtrate, concentrating to obtain Radix Puerariae extractum, adding water, adjusting pH to 3-5 with an acid, then adjusting pH to 6-9 with one or more of a base or sodium carbonate or sodium hydrogen carbonate, filtering, then extracting with one or more solvents of C3-C6 lower alcohols or C3-C8 lower ketones, such as hexone, or C2-C8 lower esters, drying by evaporation to obtain a solid, adding water, then extracting with one or more solvents of C3-C6 lower alcohols or C3-C8 lower ketones, such as hexone, or C2-C8 lower esters, concentrating, standing, filtering, performing recrystallization one or more times with one or more crystallization solvents of water, C1-C6 lower halogenated hydrocarbons, C3-C8 lower ketones, such as acetone, C2-C8 lower esters, C1-C6 lower alcohols, such as methanol, ethanol, isopropanol, C1-C6 lower fatty acids, tetrahydrofuran, acetonitrile, filtering, washing with water, and drying the resultant solid to obtain a crystalline puerarin hydrate;

Method B:

To Radix Puerariae extractum or Radix Puerariae total flavonoids or Radix Puerariae water extract concentrate or Radix Puerariae alcohol-water extract concentrate, adding one or more of water or C1-C6 lower alcohols, filtering with microporous filtration membrane (including ceramic membrane), passing through neutral alumina chromatography column, silica gel chromatography column and macroporous resin, or polyamide chromatography column or glucose gel resin chromatography column, eluting with one or more of water or C1-C6 lower alcohols, C1-C6 lower halogenated hydrocarbons, concentrating or concentrating with ultrafiltration membrane, standing, filtering, performing recrystallization one or more times with one or more crystallization solvents of water, C1-C6 lower halogenated hydrocarbons, C3-C8 lower ketones, such as acetone, C2-C8 lower esters, C1-C6 lower alcohols such as methanol, ethanol, isopropanol, C1-C6 of lower fatty acids, tetrahydrofuran, acetonitrile, filtering, washing with water, drying the resultant solid to obtain a crystalline puerarin hydrate;

Method C:

To Radix Puerariae extractum or Radix Puerariae total flavonoids or Radix Puerariae water extract concentrate or Radix Puerariae alcohol-water extract concentrate, adding one or more of water or C1-C6 lower alcohols, subjecting to filtration or concentration one or more times with one or more of microporous filtration membrane, hollow fiber membrane, ceramic membrane, ultrafiltration membrane, nanofiltration membrane, performing recrystallization one or more times with a crystallization solvent of one or more of water, C1-C6 lower halogenated hydrocarbons, C3-C8 lower ketones, such as acetone, C2-C8 lower esters, C1-C6 lower alcohols such as methanol, ethanol, isopropanol, C1-C6 of lower fatty acids, tetrahydrofuran, acetonitrile, filtering, washing with water, drying the resultant solid to obtain a crystalline puerarin hydrate;

Method D:

To Radix Puerariae extractum or Radix Puerariae total flavonoids or Radix Puerariae water extract concentrate or Radix Puerariae alcohol-water extract concentrate, subjecting to filtration or concentration one or more times with one or more of hollow fiber membrane, ceramic membrane, ultrafiltration membrane, nanofiltration membrane, performing recrystallization one or more times in a magnetic field with a magnetic flux of 0.1-5 Tesla with a crystallization solvent of one or more of water, chloroform, C3-C6 lower ketones, such as acetone, C2-C8 lower esters, C1-C6 lower alcohols such as methanol, ethanol, isopropanol, C1-C6 of lower fatty acids, tetrahydrofuran, acetonitrile, filtering, washing with water, drying to obtain crystalline puerarin hydrate; and

Method E:

Subjecting an anhydrous puerarin to hydration or recrystallization.

Different steps of the above methods A-D can be used alternatively to prepare crystalline puerarin hydrate.

In the above preparation methods, said drying method can be that: the final product is dried under conditions of different temperatures (e.g., 20-100° C.), drying time (1 hour to several days), optionally with other drying agents (including silica gel, phosphorus pentoxide, anhydrous calcium chloride, anhydrous sodium sulfate, etc.), or by means of normal pressure or reduced pressure, wherein the drying temperature is preferably 80° C. or below.

Further another aspect of the present invention relates to a composition comprising the puerarin hydrate. Specifically, said composition can be a freeze-dried powder injection, a small volume injection, a large volume infusion solution, a tablet, a capsule, a granule, an eye drops, or an ophthalmic gel.

In one embodiment of the present invention, said pharmaceutical composition further comprises a cyclodextrin or a cyclodextrin derivative, wherein the mass ratio or weight ratio of crystalline puerarin hydrate to cyclodextrin or cyclodextrin derivative is 1:5 to 1:60, preferably, the cyclodextrin or cyclodextrin derivative is one or more selected from the group consisting of 2-hydroxypropyl-β-cyclodextrin, 3-hydroxypropyl-β-cyclodextrin, and sulfobutyl ether-β-cyclodextrin (SBE-β-CD).

The crystalline puerarin hydrate together with cyclodextrin can form a water soluble inclusion (also called complex), which can be used for manufacturing more stable preparations.

The tablets, capsules, granules used for manufacturing preparations for enteral administration can comprise a pharmaceutically acceptable filler, such as starch, modified starch, lactose, sucrose, microcrystalline cellulose, cyclodextrin, sorbitol, mannitol, calcium phosphate, amino acids, etc.; a pharmaceutically acceptable disintegrant, such as starch, modified starch, microcrystalline cellulose, sodium carboxymethyl starch, cross-linked polyvinylpyrrolidone, low-substituted hydroxypropyl cellulose, hydroxypropyl starch, sodium carboxymethylcellulose, guar gum, methylcellulose, surfactant; a pharmaceutically acceptable wetting agent and adhesive, such as gelling starch, methyl cellulose, sodium carboxymethyl cellulose, ethyl cellulose, polyvinylpyrrolidone, alginic acid and salts thereof; a pharmaceutically acceptable lubricant and glidant, such as one or more of stearic acid, magnesium stearate, zinc stearate, superfine silica gel, talc powder, polyethylene glycol 2000-8000, magnesium dodecylsulfate, etc.; a pharmaceutically acceptable sweetening agent, such as sodium cyclamate, aspartame, saccharin sodium, rebaudioside, xylitol, sorbitol, xylose, lactose, glycyrrchizin, sucrose, sucralose, etc.; a pharmaceutically acceptable suspension stabilizer, such as xanthan gum, guar gum, methylcellulose, ethylcellulose, sodium carboxymethylcellulose, sodium carboxymethylstarch, polyethylene glycol 2000-8000, surfactant, alginic acid and salts thereof, etc.; wherein the adhesive can be one or more of polyvinylpyrrolidone, methylcellulose, ethylcellulose, hydroxypropylmethylcellulose, pregelatinized starch, starch, gelatin, arabic gum; the adhesive can be a solvent formed with water or ethanol or a mixture thereof.

As for eye drops of crystalline puerarin hydrate, the weight parts of the components can be: crystalline puerarin hydrate 0.2-2, cosolvent 1-10, antioxidant 0.02-0.5, metal complexing agent 0.01-0.2, osmotic pressure regulating agent 0.5-10, preservative 0.002-0.4, water 10-100 parts, with further addition of a suitable amount of pH regulating agent.

The method for manufacturing the eye drop of crystalline puerarin hydrate can comprise the steps: adding the puerarin hydrate to an appropriate amount of water for injection, adding cosolvent, stirring, dissolving, adding antioxidant, adding preservative, osmotic pressure regulating agent, stabilizer, pH regulating agent, water, stirring until well-combined, forming a solution form, adjusting pH to 5.0-7.0, filtering by means of microporous filtration membrane or ultrafiltration etc., assaying, sub-packing under sterile conditions to sterilized clean plastic eyedrops bottles.

As for ophthalmic gel of crystalline puerarin hydrate, the weight parts of the components in per 100 parts of gel can be: crystalline puerarin hydrate 0.2-2, gel matrix 0.05-1, osmotic pressure regulating agent 1-8, antioxidant 0.01-0.5, metal complexing agent 0.01-0.2, preservative 0.002-0.4, balanced with water, with further addition of a suitable amount of pH regulating agent.

The method for manufacturing ophthalmic gel of puerarin hydrate can comprise the steps: mixing the puerarin hydrate with 50-95% matrix evenly, wherein the matrix can be one or more of water, ethanol, glycerol, triethanolamine, glycogelatin, methylcellulose, hydroxypropylcellulose, carboxymethylcellulose, xanthan gum, polyethylene glycol 200-8000 (including: PEG200, PEG300, PEG400, PEG600, PEG800, PEG1000, PEG1540, PEG4000, etc.), poloxamers (can be Poloxamer-188, Poloxamer-237, Poloxamer-338, Poloxamer-407), polyvinylpyrrolidone, semi-synthetic stearate, water soluble monoglycerides, carbomers (such as carbomer 931, 934, 940, 974, AA-1, 1342, etc.), and can comprise a pharmaceutically acceptable bacteriostatic agent and stabilizer, and a pharmaceutically acceptable pH regulating agent. During manufacturing, the gel matrix of carbomer or glycogelatin, methylcellulose, hydroxypropylcellulose, carboxymethylcellulose, xanthan gum, poloxamer, polyethylene glycol, and so on can be dispersed in water, added with glycerol, heated with water bath, stirring homogeneously, added with formula amount of puerarin hydrate, osmotic pressure regulating agent, antioxidant, pharmaceutically acceptable bacteriostatic agent and stabilizer, stirring, adjusting pH to 5.0-7.0 with pharmaceutically acceptable pH regulating agent, adding with water to full dose, stirring homogeneously, filtering, sterilizing, sub-packing to obtain. Or, the following method is used:

The puerarin hydrate, osmotic pressure regulating agent, antioxidant, pharmaceutically acceptable bacteriostatic agent and stabilizer, pharmaceutically acceptable pH regulating agent are mixed for dissolution, filtered by means of microporous filtration membrane or ultrafiltration etc., the filtrate is added to the gel matrix which is dispersed in water for injection, stirred, mixed homogeneously, adjusted pH to 5.0-7.0, added with water to full dose, stirred homogeneously, sterilized, and sub-packaged to obtain ophthalmic gel of puerarin hydrate.

The method for manufacturing freeze-dried powder injection comprises the steps: taking the puerarin hydrate, adding with a pharmaceutically acceptable cosolvent, freeze-drying supporting agent or excipient, adding with water for injection and stirring to dissolve, adjusting pH to 4.0-7.5 with a pharmaceutically acceptable acid or base, adding with active carbon 0.005-0.5% (W/V) and stirring for 15-45 min, filtering, adding with water, filtering under aseptic conditions, sub-packaging by 50-600 mg/bottle, freeze-drying, plugging, to obtain the finished product.

The pharmaceutically acceptable freeze-drying supporting agent or excipient can comprise one or more of lactose, glucose, mannitol, sorbitol, xylitol, dextran, ascorbic acid, amino acid, glycine, taurine, sodium dihydrogen phosphate, disodium hydrogen phosphate, sodium deoxycholate.

The method for manufacturing the small volume injection of puerarin hydrate comprises the steps: adding to the puerarin hydrate water for injection and pharmaceutically acceptable additives, such as a pharmaceutically acceptable cosolvent, a pharmaceutically acceptable pH regulating agent, a pharmaceutically acceptable antioxidant, an inert gas, filtering, sterilizing to obtain a sterilized small volume injection, of which the specification is 50-800 mg/ampule with pH 3.5-7.5.

The method for manufacturing the infusion solution of puerarin hydrate comprises the steps: adding to the puerarin hydrate water for injection and pharmaceutically acceptable additives, such as a pharmaceutically acceptable cosolvent, a pharmaceutically acceptable isoosmotic regulating agent, a pharmaceutically acceptable pH regulating agent, a pharmaceutically acceptable antioxidant, an inert gas, filtering, sterilizing to obtain a sterilized large volume injection, of which the specification is 100-800 mg/bottle with pH 3.5-7.5.

When manufacturing the preparations of the puerarin hydrate of the present invention, the used pharmaceutically acceptable cosolvent or solubilizing agent can be one or more of water, ethanol, propylene glycol, glycerol, 1,3-butanediol, Tween 20-80, polyethylene glycol 200-1000, polyvinylpyrrolidone, sodium dodecylsulfate, pharmaceutically acceptable amides (such as nicotinamide, thiourea, urethane, etc.), amine compounds (such as ethylenediamine, diethylamine, diethanolamine, triethanolamine, methylglucamine, ethylglucamine, etc.), basic amino acids (L-, D- or racemic arginine, lysine, histidine, citrulline, etc.), cyclodextrin, in which cyclodextrin is preferably selected from the group consisting of β-cyclodextrin, 2-hydroxypropyl-β-cyclodextrin (2-HP-β-CYD), 3-hydroxypropyl-β-cyclodextrin (3-HP-β-CYD), sulfobutyl ether-β-cyclodextrin (SBE-β-CD), etc.

The pharmaceutically acceptable bacteriostatic agent can be one or more of benzalkonium chloride, benzalkonium bromide, benzylethylamine, Xibailin ( [symbol] ), ethanol, phenylmercuric nitrate, thiomersal, benzyl alcohol, phenethyl alcohol, phenoxyethanol, nautisan, domiphen bromide, parabens (methyl paraben, ethyl paraben, butyl paraben, etc.), sorbic acid or a pharmaceutically acceptable salt thereof.

The pharmaceutically acceptable pH regulating agent can be a pharmaceutically acceptable inorganic acid or organic acid, inorganic base or organic base, and can also be a Lewis acid or base in wider sense, and can comprise one or more, and can be one or more of hydrochloric acid, phosphoric acid, propionic acid, acetic acid and acetates, such as sodium acetate etc., lactic acid and pharmaceutically acceptable lactates, pharmaceutically acceptable citrates, sodium carbonate, sodium hydrogen carbonate, potassium hydrogen carbonate, sodium hydroxide, potassium hydroxide, phosphates, tartaric acid and pharmaceutically acceptable salts thereof, borax, boric acid, succinic acid, hexanoic acid, adipic acid, fumaric acid, maleic acid, trishydroxymethylaminomethane, diethanolamine, ethanolamine, isopropanolamine, diisopropanolamine, 2-amino-2-(hydroxymethyl)-1,3-propandiolamine, 1,2-hexanediamine, N-methylglucamine, diisopropylamine and salts thereof, polyhydroxylcarboxylic acids and pharmaceutically salts thereof, such as glycuronic acid, glucanic acid, lactobionic acid, malic acid, threonic acid, glucoheptonic acid, taurine, amino acids and amino acid salts, phosphate buffers, boric acid buffers, etc.

The pharmaceutically acceptable antioxidant and stabilizer (including complexing agent) can be one or more of sulfinic acid, sulfites, bisulfites, pyrosulfites, hyposulfites, thiosulfates, organic sulfur compounds such as thiourea, glutathione, dimercaprol, thioglycolic acid and salts thereof, thiolactic acid and salts thereof, thiodipropionic acid and salts thereof, phenolic compounds, such as gallic acid and salts thereof, caffeic acid, caffeiates, ferulic acid, ferulates, di-tert-butyl-p-phenol, 2,5-dihydroxybenzoic acid, 2,5-dihydroxybenzoates, phenol or derivatives thereof, salicylic acid and salts thereof; amino acids and salts thereof; ascorbic acid and ascorbates, isoascorbic acid and isoascorbates, nicotinamide, tartaric acid, nitrates, phosphates, pharmaceutically acceptable acetates, citrates, taurine, EDTA and EDTA salts, such as EDTA disodium, EDTA tetrasodium, N-di(2-hydroxyethyl)glycine, cyclodextrin, in which cyclodextrin is preferably selected from the group consisting of β-cyclodextrin, 2-hydroxypropyl-β-cyclodextrin (2-HP-β-CYD), 3-hydroxypropyl-β-cyclodextrin (3-HP-β-CYD), sulfobutyl ether-β-cyclodextrin (SBE-β-CD), etc.

The pharmaceutically acceptable osmotic pressure regulating agent can be one or more of glucose, fructose, xylitol, sorbitol, mannitol, invert sugar, maltose, dextran, sodium chloride, potassium chloride, sodium lactate, boric acid etc.

The removal of pyrogens and bacteria can be performed by adding 0.005-3% of active carbon relative to formula dose to remove pyrogens, using microporous membrane and thermocompression to remove bacteria, or by using ultrafiltration to remove bacteria and pyrogens. In ultrafiltration method, ultrafilter can be plate type, coil type, tube type, hollow fiber type or round box type, preferably, coil type and hollow fiber type ultrafilters. A filtration membrane with a molecular weight cut off of 50,000 to 300,000 can be used to remove most of pyrogens and bacteria, then a filtration membrane with a molecular weight cut off of 1,000 to 30,000, preferably a filtration membrane with a molecular weight cut off of 4,000 to 20,000, is used to remove the residual pyrogens.

Further another aspect of the present invention relates to use of the puerarin hydrate of the present invention in the manufacture of an angiotensin converting enzyme inhibitor.

Since 1980s, because of the extensive application of angiotensin converting enzyme inhibitor (ACEI) in clinical cardiovascular diseases, and the discovery and studying of local rennin-angiotensin system (RAS), people realized that RAS, especially cardiovascular local RAS, plays a very important role in many cardiovascular diseases. Angiotensin converting enzyme (ACE) is situated in RAS center, and is a rate-limiting enzyme of RAS activity, and can convert angiotensin I (Ang I) into angiotensin II (Ang II). It has been confirmed now that Ang II has functions of coronary artery constriction and positive myocardiac strength; can directly or indirectly result in vascular smooth muscle constriction by promoting release of catecholamine; can stimulate proliferation of smooth muscle cells, promote hypertrophy of cardiac muscle cells; participate ischemia-reperfusion injury, induce arrhythmia. ACE can further hydrolyze bradykinin, and the latter is a vascular dilation substance.

Under normal physiological conditions, angiotensin II in vivo is an important regulatory substance to maintain blood pressure in circulation and morphology of heart and vessels. Under pathological state of hypertension or insulin resistance, hypertension or insulin level increases, which promote the activity of rennin-angiotensin system (RAS) and result in excessive angiotensin II generated in plasma and local tissues. The excessive angiotensin II promotes the phosphorylation of the light chain of myosin in smooth muscle cells, which results in direct vascular smooth muscle constriction and stimulating organism to generate excessive active oxygen, inactivating nitric oxide (NO) released from endothelial cells to decrease NO concentration, and meanwhile increasing activity of sympathetic nerve, so that peripheral circulation resistance increases to elevate blood pressure.

On the other hand, excessive Ang II destroys insulin signal transduction and influences on hemodynamics, reduces glucose transport via skeletal muscle cells, promotes insulin resistance, and forms vicious cycles. In addition, excessive Ang II stimulates expression of transforming growth factor-b1 (TGF-b1) and inhibitor of type I plasminogen activator, and the formation of these substances closely relates to the occurrence of many severe complications of insulin resistance, such as high blood pressure, heart remodeling, and atherosclerosis.

Some researches show that ACEI has many pharmacologic actions, mainly comprising: 1) inhibiting angiotensin II converting enzyme system in circulation; 2) inhibiting tissues and vascular angiotensin system; 3) reducing norepinephrine released from peripheral neurons; 4) reducing endothelin generated in endothelial system; 5) increasing bradykinin and prostacycline formation for vasodilatation; 6) reducing secretion of aldosterone so as to reduce water-sodium retention.

Angiotensin converting enzyme inhibitor via the above actions can bring about the following effects: reducing blood pressure; inverting heart and vascular structure, reducing hypertrophy and weight of left ventricular, reducing the ratio of media thickness/diameter of lumen of resistance vessels, so as to reduce hyperplasia and hypertrophy of cardiac muscle cells after hypertension, myocardial infarction and cardiac dysfunction, to improve prognosis after myocardial infarction, to reduce accident occurrence rate and death rate, to reduce or prevent injure of hypertension target organs, to treat congestive heart failure and cardiac dysfunction, to reduce congestive heart failure occurrence rate and death rate, to increase vascular compliance, to reduce formation of atherogenesis and to combat ischemia; to reduce insulin resistance, to improve insulin sensitivity; to have renoprotective effects, especially for diabetic hypertension patients, since afferent glomerular arteriole has an increased pressure, the entered blood flow is great, which inevitably results in high filtration state of glomerular, decrease of angiotensin II generation, so that nephroprotection is achieved.

The pharmacodynamic experiments in the Examples of the present invention show that crystalline puerarin hydrate not only has antiarrythmic function, but also has inhibition effects on ACE activity, suggesting crystalline puerarin hydrate can reduce generation of Ang II and decomposition of bradykinin. Hence, the use of crystalline puerarin hydrate contributes to the prevention of arrhythmia, hypertension, congestive heart failure, myocardial ischemia and myocardial infarction etc., as well as ventricular remodeling, myocardial hypertrophy, hyperlipidemia, non-diabetic nephropathy, type I diabetic nephropathy, and proteinuria caused by the reasons above, and protection of renal function, prevention of occurrence and development of atherosclerosis.

Further another aspect of the present invention relates to use of the puerarin hydrate in the manufacture of a β-receptor blocking agent.

The crystalline puerarin hydrate can be dissolved in a suitable solvent, or lose water of crystallization and release puerarin in vivo, or directly act on a corresponding target organ or site in vivo. Puerarin can block isoprenaline so that femoral vein of a cat exhibits dose dependency in dilatation caused by methoxamine induced constriction, which is similar to the function of Propranolo, and it suggests the effect of puerarin on ex vivo vessel of cat is that of β-receptor blocking agent. In addition, puerarin also has significant antagonistic action on ex vivo atrial muscle β1 receptor of guinea pigs, while a relatively great concentration is required to show antagonistic action on trachea strip β2 receptor of guinea pigs, which suggests its selectivity on β1 receptor is higher than that on β2 receptor. Animal tests exhibit puerarin is a β receptor antagonist separately in levels of ex vivo organs and whole animals. It is known that effector of β receptor is adenylate cyclase, which can be activated with a receptor agonist and inhibited with an antagonist. Puerarin can bind to β receptor and completely inhibit activation function of adrenalin on adenylate cyclase.

The blocking function of puerarin on β receptor renders the crystalline puerarin hydrate to show effects and uses of antihypertension, reducing intraocular pressure of glaucoma, and combating arrhythmia.

Puerarin has a certain antihypertensive effect in animals with normal blood pressure and with high blood pressure. The injection of puerarin can result in rapid dose-dependent decrease of blood pressure in anesthetized dogs, and this effect can be maintained for some time. Intraperitoneal injection of puerarin can significantly reduce blood pressure and heart rate in conscious spontaneously hypertensive rats.

Puerarin can reduce intraocular pressure of ocular hypertension rabbit models induced by sub-conjunctival injection of dexamethasone, and shows a correlation between action strength and drug concentration. In addition, puerarin eye drops can inhibit intraocular pressure increase caused by rapid injection of glucose into ear vein of rabbit, which is similar to the function of timolol eye drops, and it suggests that puerarin eye drops are an ideal antiglaucomatous drug. Some clinic experiments show that puerarin has function of reducing intraocular pressure in treatment of most constitutional open angle glaucoma, angle closure glaucoma, secondary glaucoma; puerarin can also improve microcirculation and retina function to prevent cataract.

Instillation of puerarin can combat mesentery microcirculation dysfunction caused by epinephrine in anesthetized mice; when patients with retinal arterial obstruction are treated with puerarin, the simultaneous retina fluorescence visualization shows a reduction of microcirculation time, and meanwhile, it is found that puerarin can significantly enhance visual acuity, broaden visual field. In addition, puerarin can further prevent or treat retinal arterial obstruction by improving hemorheological indexes, such as reducing whole blood specific viscosity, red cell electrophoresis, hematocrit, and fibrinogen. The injection of puerarin in treatment of sudden deafness gives good state of nail fold microcirculation and improved audition in 70% patients within 2 weeks after administration, and their hearing is improved in different degree.

Intravenous drip of puerarin in 60 patients with vertebrobasilar ischemia gives improvement in symptoms. Puerarin can inhibit atherosclerosis, promote softness of vessels, and influence TXB2, NO, ET so as to regulate cerebral blood flow, improve cerebral circulation. Puerarin can improve hemorrheologic indexes, prevent cervical osteoarthritis. Puerarin can reduce blood viscosity and red cell volume, shorten erythrocyte electrophoretic time, slow blood sedimentation, and reduce fibrinogen content.

Puerarin injection solution is used for adjunctive treatment of cervical osteoarthritis, and the results are: the treatment group exhibits a clinic total effective rate of 98%, which shows significant difference in comparison with the control group; the treatment group exhibits shortened time in the disappearance of symptoms and signs, and the detected hemorrheologic indexes show a significant decrease of whole blood viscosity, plasma viscosity, fibrinogen level and blood sedimentation level after treatment.

Puerarin can improve brain microcirculation, prevent or treat basilar-vertebral artery ischemic dizziness, treat cervical osteoarthritis, so that the crystalline puerarin hydrate would exhibit effects of improving brain microcirculation, preventing or treating basilar-vertebral artery ischemic dizziness, and treating cervical osteoarthritis.

Some researches show that intraperitoneal injection of puerarin can significantly reduce ethanol intake in rats, of which the mechanism may be related with inhibition of mitochondrial aldose reductase. In addition, it is found that puerarin is an antagonist or partial agonist of benzodiazepine receptor, and some pharmacological effects of ethanol act via benzodiazepine receptor of brain cells.

Intravenous injection of puerarin into coronary artery of anesthetized or awake dogs can both results in increase of blood flow of coronary artery and decrease of vascular resistance, which are strengthened with the increase of dose. Puerarin can reduce heart beat rate and cardiac contractility, but does not reduce blood flow of collateral coronary artery in ischemic region, so that the effects of puerarin on ischemic cardiac muscle are achieved by reducing collateral coronary resistance. In addition, it can combat acute myocardial ischemia caused by pituitrin in rats, which may be result of expanding coronary artery. Puerarin can significantly reduce formation of myocardial lactic acid during ischemia reperfusion, reduce oxygen consumption and creatine phosphokinase release amount, and improve the ultrastructure of cardiac muscle after ischemia reperfusion somewhat. Puerarin is used for the treatment of coronary heart disease, and the observation emphasizes on hemorheology, dynamic electrocardiogram, conventional lead electrocardiogram, and all the indexes indicate significant improvement in comparison with those before treatment, which shows that puerarin can effectively improve blood rheology, expand vessels, improve myocardial ischemia in patents with coronary heart disease, so that puerarin is a safe and effective drug combating myocardial ischemia.

Due to the release of crystalline puerarin hydrate in vivo or in vitro, it would exhibit activity of antagonist or partial agonist of benzodiazepine receptor, and would significantly reduce ethanol intake in rats as well; and would effectively improve blood rheology, expand vessels, improve myocardial ischemia in patents with coronary heart disease, and would be a safe and effective drug combating myocardial ischemia. This is confirmed with corresponding evidences in the experiments of the present invention.

The therapeutical effects of puerarin on diabetes and complications thereof indicate that the crystalline puerarin hydrate would exhibit corresponding therapeutical effects and would be used for manufacturing a corresponding medicament for the prophylaxis and treatment of these diseases.

In addition, the drug of the present invention has inverting effects on multidrug resistance of tumors such as stomach cancer, and can also be used for manufacturing a corresponding medicament for the treatment and prophylaxis of these diseases.

Further another aspect of the present invention relates to use of the puerarin hydrate in the manufacture of a medicament for the treatment or prophylaxis of any one of the following diseases:

hypertension, coronary heart disease, pulmonary heart disease, heart failure, angina pectoris, myocardial infarction, cardiogenic shock, arrhythmia, myocarditis, ischemic encephalopathy, cerebral infarction, cerebral thrombosis and sequela thereof, improvement of brain circulation, vertebrobasilar ischemia dizziness, lower limb deep vein postthrombotic syndrome, diabetes, diabetic complications such as diabetic nephropathy, diabetic peripheral neuropathy, retinopathy, retinal arterial obstruction, vein occlusions, sudden deafness, ocular hypertension, glaucoma, or dyslipidemia.

Further another aspect of the present invention relates to a method for the treatment or prophylaxis of any one of the following diseases, comprising a step of administering an appropriate amount of the puerarin hydrate of the present invention, or a pharmaceutical composition of the puerarin hydrate of the present invention:

hypertension, coronary heart disease, pulmonary heart disease, heart failure, angina pectoris, myocardial infarction, cardiogenic shock, arrhythmia, myocarditis, ischemic encephalopathy, cerebral infarction, cerebral thrombosis and sequela thereof, improvement of brain circulation, vertebrobasilar ischemia dizziness, lower limb deep vein postthrombotic syndrome, diabetes, diabetic complications such as diabetic nephropathy, diabetic peripheral neuropathy, retinopathy, retinal arterial obstruction, vein occlusions, sudden deafness, ocular hypertension, glaucoma, or dyslipidemia. Specifically, administration route can be injection, oral or ocular administration.

Dosage and usage: generally, for an adult, a freeze-dried powder injection or small liquid injection of 0.020-0.6 g of the puerarin hydrate of the present invention is taken and added to 20-500 ml of 0.9% sodium chloride or 5-10% glucose solution, and injected by intravenous push or instillation, 1-2 times per day; the large volume infusion solution of puerarin hydrate in 0.9% sodium chloride or 5-10% glucose can be administered via intravenous injection, and administration dose can be the same as the above; for intramuscular injection: a freeze-dried powder of 0.020-0.6 g of the drug of the present invention is dissolved in water for injection, administered via intramuscular injection, 1-2 times per day; for a child, half or less dose can be used. For administration via gastrointestinal tract, the dose can be generally 0.020-0.6 g/time, 1-3 times per day.

Usage of eyedrops: the general dose for an adult is 1-2 drops of 1% puerarin eyedrops per time, and it should be dropped in eyelids and then keep eyes closed for 3-5 min, 1-3 times per day. The administration dose for ophthalmic gel is in accordance with that of eyedrops.

### BENEFICIAL EFFECTS OF THE INVENTION

The puerarin hydrate of the present invention containing water of crystallization is more stable in storage than that without water of crystallization, convenient for storage and transportation, and has good fluidity at room temperature thereby easy for manufacturing preparations (e.g., granules or tablets). In addition, the puerarin hydrate of the present invention overcomes the drawback of deliquescent property of anhydrous puerarin, so that it is not necessary to isolate air to prevent adhesion, and has good sliding property, and the manipuility for manufacturing preparations is improved. Anhydrous puerarin has sticking phenomenon when its powder is used for direct tableting. When tableting 200 mg of powder, anhydrous puerarin has a sticking amount 1-3 times or higher than that of puerarin monohydrate.

### BRIEF DESCRIPTION OF THE DRAWINGS

FIG. 1: thermogram of puerarin monohydrate.

FIG. 2: thermogram of puerarin hydrate.

FIG. 3: thermogram of puerarin monohydrate.

FIG. 4: thermogram of puerarin 0.5 hydrate.

FIG. 5: X-ray diffractogram of puerarin monohydrate powder.

FIG. 6: X-ray diffractogram of puerarin monohydrate powder.

FIG. 7: X-ray diffractogram of puerarin 0.5 hydrate powder.

### SPECIFIC MODELS FOR CARRYING OUT THE INVENTION

Some embodiments of the present invention are illustrated in conjunction with the following examples, but those skilled in the art would understand that these examples are merely used to illustrate the invention, and should not be deemed as to limit the scope of the present invention. The specific techniques and conditions that are not specifically annotated in the examples are those techniques and conditions as described in documents (e.g., by referring to J. Sambrook, et al, “Molecular Cloning: A laboratory Manual”, translated by HUANG Peitang et al, 3^{rd} Edition, Science Press) or are performed according to product specifications. All reagents or instruments which manufacturers are not given are conventional products commercially available on the market.

### Example 1

### Preparation of Sample 1 of Puerarin Hydrate

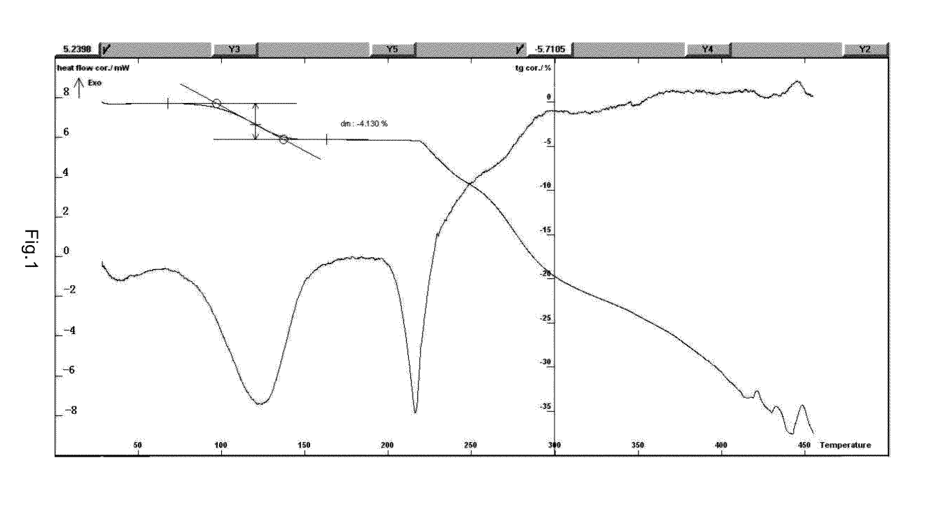

In a reaction container, 80 g of dry Radix Puerariae powder was extracted for 3 times under refluxing with 400 ml of 80% ethanol aqueous solution, all extracting solutions were combined, crude filtered, then filtered with 0.22 μm microporous filtration membrane, the filtrate was concentrated to obtain Radix Puerariae extractum, which was added with water, adjusted pH to 3-5 with diluted hydrochloric acid, then adjusted pH to 6-7 with sodium hydrogen carbonate, filtered, then extracted with n-butanol and hexone as the solvent, the extracting solution was dried by evaporation to obtain a solid, the solid was recrystallized for 3 times with water, methanol, formic acid, acetonitrile as the crystallization solvent, filtered, washed with water, the solid was dried at about 80° C. for 4 h to obtain 1.8 g of off-white crystalline powder; melting point: 208.4-212.5° C. decomposition (not calibrated), ultraviolet spectra (the sample was added with ethanol to form a solution having a concentration of 10 μg/ml): λₘₐₓ 250 nm, [α]_{D}²¹+18.14° C. (c=1, methanol), ESI-MS: m/z: 417, 399, 363; infrared spectrum: v^{KBr}ₘₐₓ cm⁻¹vKBrₘₐₓ cm⁻¹ 3383, 3229, 2900, 1913, 1632, 1607, 1568, 1515, 1448, 1396, 1273, 1236, 1208, 1175, 1104, 1059, 1008, 890, 838, 797, 748, 610, 542; water content as measured by Karl Fischer method was 4.19%, TG-DTA: platform weight loss was about 4.13%, which was within the error range of the result that the sample contained one water of crystallization (theoretical value: 4.15%) (see: FIG. 1); element analysis: measured values: C, 57.99; H, 5.26. theoretical values: C, 58.06; H, 5.10.

### Example 2

### Preparation of Sample 2 of Puerarin Hydrate

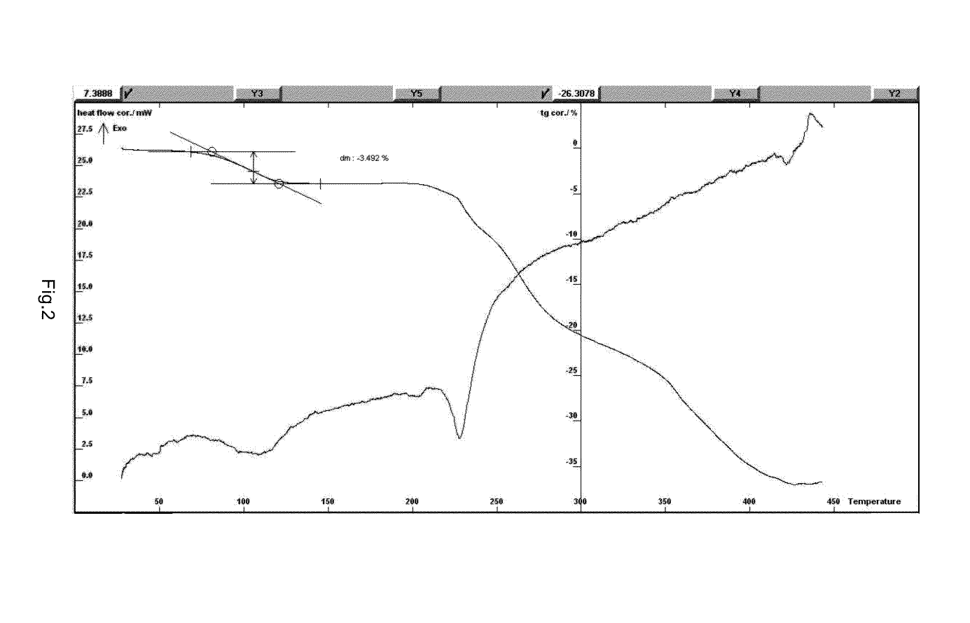

30 g of Radix Puerariae extractum or Radix Puerariae total flavonoids was added with water, heated to 50-70° C., filtered with 0.15-0.24 μm ceramic membrane, the filtrate was passed through neutral alumina chromatography column, eluted, absorbed with D101 macroporous resin, washed with water, detected with TLC, then eluted with 30-75% ethanol aqueous solution until elution was completed, filtered, reduced pressure concentrated, stood, recrystallized twice using water, acetone, ethanol, acetonitrile as the crystallization solvent, stood, filtered, washed with water, the resultant solid was dried at about 80° C. for 4-6 h to obtain 6.6 g of off-white solid, melting point: 227.8-231.8° C. (not calibrated), ultraviolet spectrum: λ^{CH3OH}ₘₐₓ 250 nm; [α]_{D}²¹+18.14° C. (c=1, methanol), ESI-MS: m/z: 417, 399, 363; infrared spectrum: v^{KBr}ₘₐₓ cm⁻¹ 3379, 3229, 2899, 1913, 1631, 1607, 1569, 1515, 1448, 1396, 1271, 1237, 1209, 1175, 1103, 1059, 1008, 890, 838, 797, 749, 610, 543; water content as measured by Karl Fischer method was 4.01%, TG-DTA: platform weight loss was about 3.49%, which was within the error range of the result that the sample contained one water of crystallization (theoretical value: 4.15%) (see: FIG. 2); element analysis, measured values: C, 58.13; H, 5.21. theoretical values: C, 58.06; H, 5.10.

### Example 3

### Preparation of Sample 3 of Puerarin Hydrate

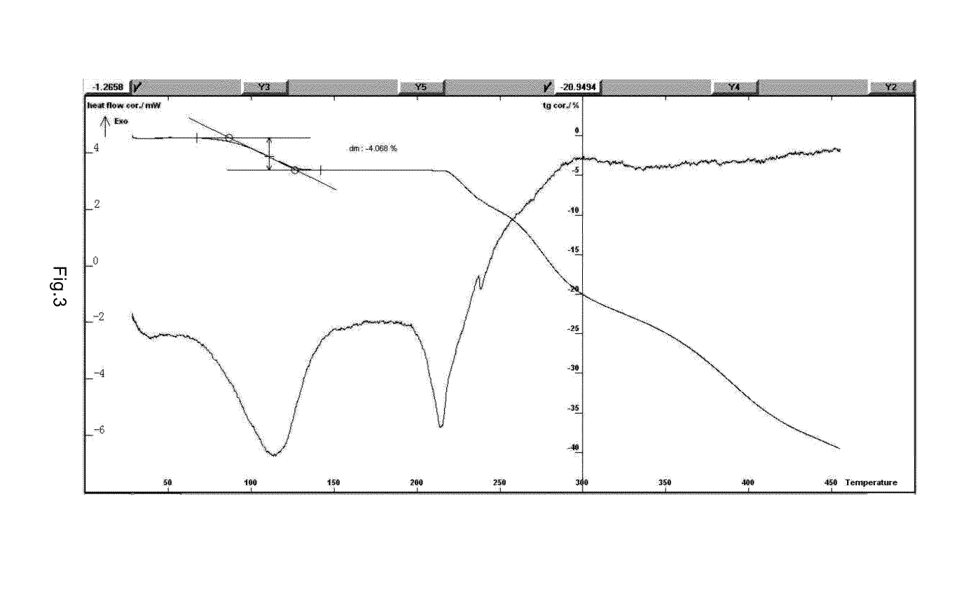

90 g of dry Radix Puerariae powder of 40 mesh was added with 400 ml of water and extracted under ultrasonic waves for 3 times, all extracting solutions were combined, crude filtered, then filtered with 0.22 μm microporous filtration membrane or ceramic membrane, the filtrate was then filtered at 25-65° C. and 0.15-2 MPa (the pressure was regulated dynamically or increased with the proceeding of the experiment) using an ultrafiltration membrane with a molecular weight cut off of 4000-50000 (polysulfone hollow fiber membrane module, fiber aperture: 0.011 μm, inner diameter: 1.1 mm), the filtrate was then filtered with nanofiltration membrane with a molecular weight cut off of 200 or more, concentrated, dissolved with 90% methanol aqueous solution, added with acetic acid, acetonitrile as the crystallization solvent, stood, crystallized, suction filtered, washed with water; as above method, recrystallized for 3 times using methanol, water, acetic acid, acetonitrile as the crystallization solvent, the resultant solid was dried at about 80° C. for 4-6 h to obtain 1.6 g of off-white crystalline solid, melting point: 215.5-218.6° C. (not calibrated), ultraviolet spectrum: λ^{CH3OH}ₘₐₓ 250 nm, [a]_{D}²¹ +18.14° C. (c=1, methanol), ESI-MS: m/z: 416, 399, 360, 350, 297, 267, 254; infrared spectrum: v^{KBr}ₘₐₓ cm⁻¹ 3380, 1625, 1587, 1515, 1446; water content as measured by Karl Fischer method was 4.21%, TG-DTA: platform weight loss was about 4.07%, which was within the error range of the result that the sample contained one water of crystallization (theoretical value: 4.15%) (see: FIG. 3); element analysis, measured values: C, 58.15; H, 5.02. theoretical values: C, 58.06; H, 5.10.

### Example 4

### Preparation of Sample 4 of Puerarin Hydrate

90 g of dry Radix Puerariae powder of 20-60 mesh was added with 400 ml of water and extracted under ultrasonic waves for 3 times, the extracting solutions were combined, crude filtered with sand type pump, then filtered with 0.22 μm microporous filtration membrane or ceramic membrane, the filtrate was then filtered at 25-65° C. and 0.15-2 MPa (the pressure was regulated dynamically or increased with the proceeding of the experiment) using an ultrafiltration membrane with a molecular weight cut off of 3000-50000 (polysulfone hollow fiber membrane module, fiber aperture: 0.011 μm, inner diameter: 1.1 mm), the filtrate was then filtered with nanofiltration membrane with a molecular weight cut off of 200 or more, concentrated, dissolved in 90% methanol aqueous solution, added with formic acid, acetic acid and water as the crystallization solvent, stood, crystallized, suction filtrated, washed with water; as above method, methanol, water, acetic acid and acetonitrile were used as the solvent to perform recrystallization for 3 times, the resultant solid was dried at about 50° C. for 4-6 h to obtain 1.6 g of off-white crystalline solid, then dried at about 50° C. for 2-4 h to obtain off-white crystalline powder, which was easy to dissolve in water, melting point: 226.5° C. (decomposition, not calibrated), ultraviolet spectrum: λ^{CH3OH}ₘₐₓ 250 nm, [α]_{D}²¹ +18.14° C. (c=1, methanol), ESI: m/z: 416[M]⁺, 399, 360, 350, 297, 267, 254; infrared spectrum: v^{KBr}ₘₐₓ cm⁻¹ 3378, 1624, 1585, 1516, 1446, water content as measured by Karl Fischer method was 5.22%, thermoanalysis TG-DTA: platform weight loss before reaching 150° C. was about 5.32%, which was within the error range of the result that the sample contained 1.25 water of crystallization (theoretical values: 5.13%).

### Example 5

### Preparation of Puerarin 0.5 Hydrate (Sample 5)

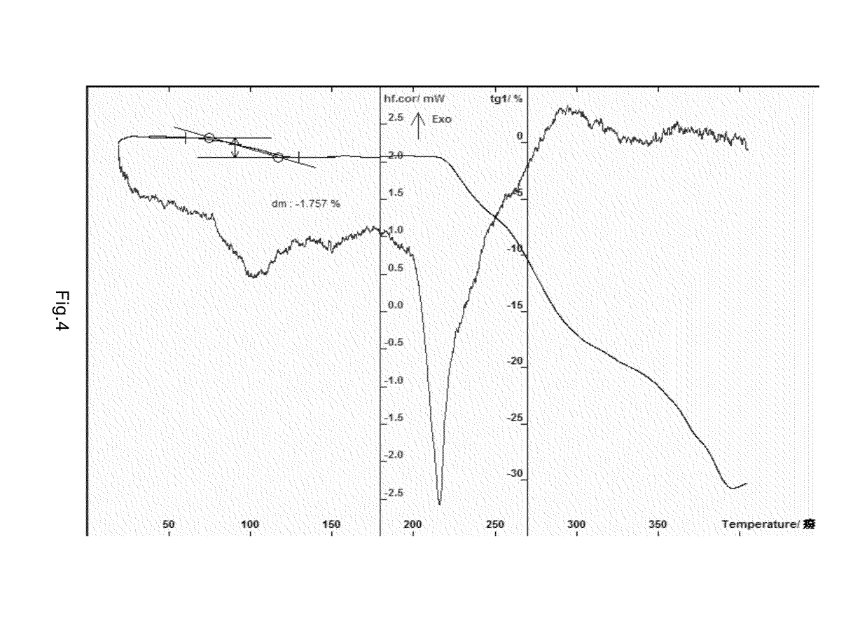

In a reaction container, 40 g of Radix Puerariae total flavonoids was added with 500 ml of water, adjusted pH to 6-7 with sodium hydrogen carbonate, dissolved, filtered with 0.22 μm microporous filtration membrane, the filtrate was adjusted pH to 3-5 with diluted hydrochloric acid, then adjusted pH to 6-7 with sodium carbonate, filtered with 0.22 μm microporous filtration membrane, passed through silica gel chromatography, washed with water, the eluant was extracted with n-butanol and hexone as the solvent, the extracting solution was dried by evaporation to obtain a solid, water, methanol and acetonitrile were used as the crystallization solvent to perform recrystallization for 3 times, filtrated, washed with water, the resultant solid was dried at about 95° C. for about 6 h to obtain 1.3 g of off-white crystalline powder; melting point: 207° C., yellow discoloration (not calibrated), ultraviolet spectrum: λ^{C2H5OH}ₘₐₓ 250 nm (the sample was added with ethanol to form a solution having a concentration of 10 μg/ml), ESI-MS: m/z: 417, 399, 381, 363; infrared spectrum: v^{KBr}ₘₐₓ cm⁻¹ 3368, 3229, 2900, 1632, 1607, 1567, 1514, 1448, 1396, 1273, 1236, 1209, 1103, 1059, 1008, 892, 837, 797, 749, 610, 543; water content as measured by Karl Fischer method was 2.32%, thermoanalysis: platform weight loss was about 1.76%, which was within the error range of the result that the sample contained 0.5 water of crystallization (theoretical values: 2.11%) (See: FIG. 4); element analysis, measured values: C, 59.02; H, 5.17. theoretical values: C, 59.25; H, 4.98.

### Example 6

### Preparation of Injection of Puerarin Hydrate

20 g of sample 1 of Example 1 was added with 10 g of arginine, 2.0-5 g of mannitol or xylitol, added with 400 ml of fresh water for injection, stirred to dissolve, adjusted pH to 4.0-6.8 with citric acid or sodium citrate, added with activated carbon 0.01-0.5% (W/V), stirred for 15-45 min, filtered, then filtered with 0.22 μm microporous filtration membrane or filtered with ultrafiltration membrane with a molecular weight cut off of 3000-8000, subpackaged in 100, 200 mg/bottle or 300 mg/bottle or 400 mg/bottle or 600 mg/botter (expressed in puerarin), added with plug, freeze-dried, plugged, and tested qualified to obtain the finished product.

### Example 7

### Preparation of Puerarin Hydrate Injection

20 g of sample 2 of Example 2 was added with 10 g of mannitol, 12 g of methylglucamine, 2 g of Tween 80, added with 320 ml of 40-60° C. water for injection, stirred for dissolution, adjusted pH to 4.0-6.5 with gluconic acid or sodium citrate, added with activated carbon 0.01-0.5% (W/V), stirred for 15-45 min, filtered, added with water for injection to 400 ml, then filtered with 0.22 μm microporous filtration membrane or filtered with ultrafiltration membrane with a molecular weight cut off of 2000-8000, subpackaged in 100, 200 mg/bottle or 300 mg/bottle or 400 mg/bottle or 600 mg/botter (expressed in puerarin), added with plug, freeze-dried, plugged, and tested qualified to obtain the finished product.

### Example 8

### Preparation of Puerarin Hydrate Injection

10 g of puerarin 0.5 hydrate as prepared in Example 5 was added to 90 ml of propanediol, added with 1 ml of Tween 80, stirred for dissolution, then added with 90 ml of fresh water for injection, stirred, and while stirring, added with 0.1 g of sodium pyrosulfite, 0.1 g of EDTA disodium, adjusted pH to 4.0-6.3 with 2M lactic acid and sodium lactate, added with activated carbon 0.3% (W/V), stirred for 15-45 min, filtered, measured to determine content and pH, added with water to reach a total volume of about 200 ml, filtered with 0.22 μm microporous filtration membrane, fed with nitrogen, encapsulated in 2-20 ml/bottle, sterilized, tested qualified to obtain the finished product.

### Example 9

### Preparation of puerarin hydrate injection

10 g of sample 3 of Example 3 was added to 90 ml of propanediol, then added with 2 ml of Tween 80, stirred for dissolving, added with 90 ml of fresh water for injection, stirred, and while stirring, added with 0.1 g of sodium pyrosulfite, 0.1 g of EDTA disodium, adjusted pH to 4.0-6.3 with 2M lactic acid and sodium lactate, added with activated carbon 0.5% (W/V), stirred for 15-45 min, filtered, measured to determine content and pH, added with water to reach a total volume of about 200 ml, filtered with 0.22 μm microporous filtration membrane, fed with nitrogen, encapsulated in 2-20 ml/bottle, sterilized, tested qualified to obtain the finished product.

### Example 10

### Preparation of Puerarin Hydrate Injection

10 g of sample 3 of Example 3 (expressed in puerarin), 100 ml of glycerol, 2 ml of Tween 80, 0.2 g of cysteine hydrochloride, 0.4 g of glycine, 0.5 g of taurine, 2 g of 3-hydroxypropyl-8-cyclodextrin, 0.1 g of EDTA disodium were added to 250 ml of fresh water for injection, stirred for dissolving, adjusted pH to 4.0-6.5 with 2M gluconic acid and sodium gluconate, added with activated carbon 0.5% (W/V), stirred for 15-45 min, filtered, added with water to reach a total volume of about 400 ml, filtered with 0.22 μm microporous filtration membrane, fed with nitrogen, encapsulated in 2-10 ml/bottle, sterilized, tested qualified to obtain the finished product.

### Example 11

### Preparation of Large-Volume Injection of Puerarin Hydrate

10.1 g of sample 1 of Example 1 (expressed in dry product) was completely dissolved with 4.5 L of fresh water for injection, added with 1 g of sodium pyrosulfite, 250 g of glucose, 2 g of taurine, 0.2 g of EDTA disodium, adjusted pH to 4.0-6.8 with 4M sodium dihydrogen phosphate or disodium hydrogen phosphate solution, added with 0.02-0.5% (W/V) of activated carbon relative to formula dose, heated and stirred for 15-30 min, filtered to remove the activated carbon, measured to determine content and pH, added with water to obtain 5 L of solution, fine-filtrated with 0.22 um microporous filtration membrane or filtered with ultrafiltration membrane with a molecular weight cut off of 4000-8000, subjected to semi-finished product assay, when its content, pH value and clarity were qualified, it was encapsulated in 50 ml or 100 ml or 200 ml or 250 ml glass bottles, hot-pressed and sterilized for 30 min, cooled, subjected to inspection of finished product, and packaged to obtain the product.

### Example 12

### Preparation of Puerarin Hydrate Sodium Chloride Infusion

10 g of sample 1 of Example 1 (expressed in puerarin), 45 g of sodium chloride, 1 g of L-cysteine hydrochloride, 1 g of sodium pyrosulfite, 2 g of glycine, 0.2 g of EDTA disodium were added to 4.5 L of fresh water for injection, controlled to have a temperature of 80° C. or below, stirred to dissolve completely, adjusted pH value to the range of 4.0 to 7.0 with 2M citric acid or sodium citrate, added with 0.1% of activated carbon relative to formula dose, heated and stirred for 15-30 min, filtered to remove the activated carbon, measured to determine content and pH value, added water so that the solution was of 5 L, then fine-filtered with 0.22 μm microporous filtration membrane or filtered with ultrafiltration membrane with a molecular weight cut off of 4000-8000, after its clarity and insoluble particulate matter were tested qualified, it was fed with nitrogen gas, encapsulated in 50 ml or 100 ml or 200 ml or 250 ml glass bottles, sterilized, subjected to finished product inspection, and packaged to obtain the product.

### Example 13

### Tablets of Puerarin 0.5 Hydrate (200 mg/Tablet)

Formula:

| | |
|---|---|
| puerarin 0.5 hydrate | 200 g |
| microcrystalline cellulose | 80 g |
| sodium carboxymethyl starch | 20 g |
| 10% povidone K-30 | appropriate amount |
| ethanol-water (7:3) solution | |
| magnesium stearate | 1 g |

The puerarin 0.5 hydrate as prepared according to Example 5, microcrystalline cellulose, sodium carboxymethyl starch were passed through 100 mesh sieve, added with 10% povidone K30 ethanol-water (7:3) solution to form a soft material, granulated by passing through 18-24 mesh sieve, dried, trimmed by passing through 14-20 mesh sieve, added with magnesium stearate and mixed homogeneously, tableted, inspected, and packaged.

### Example 14

### Tablets of Puerarin Monohydrate (200 mg/Tablet)

Formula:

| | |
|---|---|
| puerarin monohydrate (prepared | 200 g |
| by method of Example 2) | |
| microcrystalline cellulose | 80 g |
| sodium carboxymethyl starch | 20 g |
| 10% povidone K-30 | appropriate amount |
| ethanol-water (7:3) solution | |
| superfine silica gel | 1 g |

Puerarin monohydrate, microcrystalline cellulose, sodium carboxymethyl starch were passed through 100 mesh sieve, added with 10% povidone K-30 ethanol-water (7:3) solution to form a soft material, granulated by passing through 18-24 mesh sieve, dried, trimmed by passing through 14-20 mesh sieve, added with superfine silica gel and mixed homogeneously, tableted, inspected, and packaged.

### Example 15

### Capsules of Puerarin Monohydrate (100 mg/Capsule)

Formula:

| | |
|---|---|
| puerarin monohydrate (prepared | 100 g |
| by method of Example 3) | |
| microcrystalline cellulose | 100 g |
| lactose | 20 g |
| superfine silica gel | 2 g |

Puerarin monohydrate, microcrystalline cellulose, lactose were passed through 100 mesh sieve, added with superfine silica gel that passed through 100 mesh sieve, mixed, encapsulated in capsules.

### Example 16

### Tablets of Puerarin Monohydrate (100 mg/Tablet)

Formula:

| | |
|---|---|
| puerarin monohydrate (prepared | 100 g |
| by method of Example 1) | |
| microcrystalline cellulose | 100 g |
| lactose | 20 g |
| magnesium stearate | 2 g |

Puerarin monohydrate, microcrystalline cellulose, lactose were passed through 100 mesh sieve, added with magnesium stearate that passed through 100 mesh sieve, mixed, encapsulated in capsules.

### Example 17

### Capsules of Puerarin Monohydrate (100 mg/Capsule)

Formula:

| | |
|---|---|
| puerarin monohydrate (prepared | 100 g |
| by method of Example 4) | |
| microcrystalline cellulose | 50 g |
| lactose | 10 g |
| gelatinized starch 10% | appropriate amount |
| magnesium stearate | 2 g |

Puerarin monohydrate, microcrystalline cellulose, lactose were passed through 100 mesh sieve, added with 10% gelatinized starch to form a soft material, granulated by passing through 18-24 mesh, dried, trimmed by passing 14-20 mesh sieve, added with magnesium stearate and mixed, encapsulated in capsules.

### Example 18

### Granules of Crystalline Puerarin Hydrate (200 mg/Bag)

Formula:

| | |
|---|---|
| crystalline puerarin hydrate | 200 g |
| (prepared by method of Example 2) | |
| mannitol | 100 g |
| lactose | 20 g |
| sodium cyclamate | 2 g |
| solid edible essence | 1 g |
| xanthan gum | 2 g |
| 8% povidone K-30 ethanol-water solution | appropriate amount |

Crystalline puerarin hydrate, mannitol, lactose, sodium cyclamate, edible essence were passed through 100 mesh sieve, added with 8% povidone K-30 ethanol-water solution to form a soft material, granulated by passing through 18-24 mesh sieve, dried at 60° C. or below, added with xanthan gum that passed through 100 mesh sieve, trimmed by passing through 14-20 mesh sieve, mixed homogeneously, added with xanthan gum that passed through 100 mesh sieve, sub-packaged.

### Example 19

### Eyedrops of Puerarin Monohydrate

5.23 g of puerarin monohydrate (prepared by method of Example 1), 50 ml of glycerol, and 5 g of povidone K-30 were added to water for injection in appropriate amount, stirred for dissolution, then 0.6 g of sodium sulfite, 0.3 g of EDTA disodium, 1 ml of 5% benzalkonium chloride solution, 3.1 g of sodium chloride were added to the above solution, stirred for dissolution, adjusted pH to 6.0-7.0 with solution of sodium hydrogen phosphate and disodium hydrogen phosphate, then added with water for injection to reach a volume of 500 ml, stirred homogeneously, detected, filtered with 0.45-0.2 μm microporous filtration membrane to be clear, sub-packaged in sterilized clean eyedrops bottles, sterilized, cooled to obtain the product.

### Example 20

### Eyedrops of Puerarin 0.5 Hydrate

5.23 g of puerarin 0.5 hydrate (prepared by method of Example 5), 50 ml of glycerol, and 5 g of povidone K-30 were added to water for injection in appropriate amount, stirred for dissolution, then 0.6 g of sodium sulfite, 0.3 g of EDTA disodium, 1 ml of 5% benzalkonium chloride solution, 3.1 g of sodium chloride were added to the above solution, stirred for dissolution, adjusted pH to 6.0-7.0 with solution of sodium hydrogen phosphate and disodium hydrogen phosphate, then added with water for injection to reach a volume of 500 ml, stirred homogeneously, detected, filtered with 0.45-0.2 μm microporous filtration membrane to be clear, sub-packaged in sterilized clean eyedrops bottles, sterilized, cooled to obtain the product.

### Example 21

### Eyedrops of Puerarin Monohydrate

5.23 g of puerarin monohydrate (prepared by method of Example 3), 50 ml of glycerol, and 5 g of povidone K-30 were added to water for injection in appropriate amount, stirred for dissolution, then 0.6 g of sodium pyrosulfite, 0.3 g of EDTA disodium, 4 g of mannitol were added to the above solution, stirred for dissolution, adjusted pH to 6.0-7.0 with boric acid buffer solution, then added with water for injection to reach a volume of 500 ml, stirred homogeneously, detected, filtered with 0.45-0.2 μm microporous filtration membrane to be clear, sub-packaged in sterilized clean eyedrops bottles, sterilized, cooled to obtain the product.

### Example 22

### Ophthalmic Gel of Puerarin Monohydrate

Formula: 5.2 g of puerarin monohydrate (prepared by method of Example 1), 2 g of Carbomer 934 or Carbomer 971, 10 ml of glycerol, 6 g of mannitol, 0.1 g of EDTA disodium, 2 g of borax, 1 g of boric acid, 0.3 g of ethyl paraben, appropriate amount of sodium hydrogen phosphate and disodium hydrogen phosphate solution to regulate pH value, and water for injection to be added to reach 500 ml.

Carbomer 934 was dispersed and swollen in appropriate amount of water for injection, added with glycerol, heated with water-bath, stirred homogeneously, added puerarin hydrate, glycerol, mannitol, EDTA disodium, borax, boric acid, ethyl paraben in formula amounts to an appropriate amount of water for injection, stirred, adjusted pH to 6.0-6.7 with pH regulating agent for dissolution, the resultant mixture was added to a gel matrix dispersed in water, stirred, added water to full dose, stirred homogeneously, filtered, sterilized, sub-packaged to obtain the product.

### Example 23

### Thermoanalysis Test

The four samples of the hydrates of the present invention (separately prepared by methods of Examples 1, 2, 3, 5) were subjected to thermoanalysis (TG-DSC or TG-DTA), and it could be seen from their spectra that characteristic weight loss platforms in different and approximately horizontal step state (between 60° C. and 150° C.) had corresponding strong endothermic peaks, and thermoanalysis spectra showed puerarin monohydrate (C₂₁H₂₀O₉.H₂O), puerarin 1.25 hydrate (C₂₁H₂₀O₉.1.25H₂O), and the water contents as measured by Karl Fischer method were in consistence with the thermoanalysis results. Test conditions: Setsys 16 of Setaram Company, sample amount was about 5 mg, rate of temperature increase: 10K/min, N₂ flow rate: 50 ml/min, at room temperature to about 400° C. The test results are shown in FIG. 1-FIG. 4.

### Example 24

### X-Ray Diffraction Test

D/MX-IIIA X-ray diffract meter, diffraction angle 2θ, scanning range 3-60°, was used to test powder X-ray diffraction spectra of puerarin hydrates.

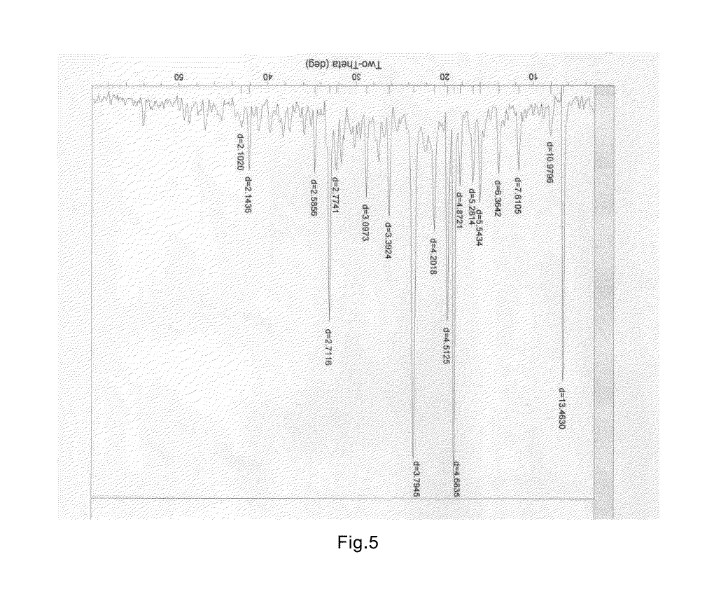

As measured by powder X-ray diffraction method, in range of diffraction angle 2θ (3-60°, the puerarin monohydrate (prepared by method of Example 1) had corresponding characteristic values at following 2θ values: about 6.6, 8.0, 11.7, 14.0, 16.0, 16.7, 18.2, 19.0, 19.7, 21.1, 23.4, 26.2, 28.8, 32.2, 34.7, 42.1. The results are shown in FIG. 5.

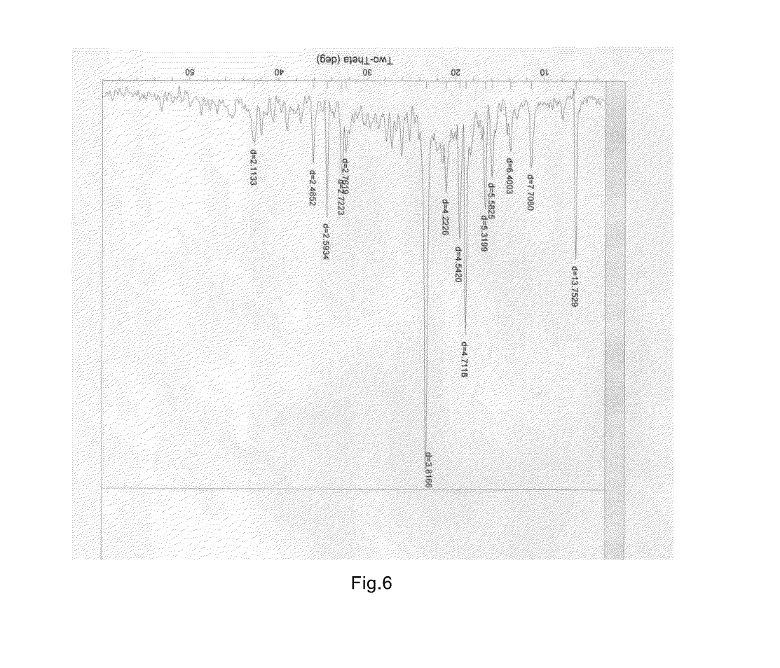

As measured by powder X-ray diffraction method, in range of diffraction angle 2θ (3-60°, the puerarin monohydrate (prepared by method of Example 2) had corresponding characteristic values at following 2θ values: about 6.4, 8.0, 11.5, 13.8, 15.7, 16.7, 18.8, 19.5, 21.0, 23.3, 26.2, 32.4, 32.9, 34.6, 36.1, 42.8. The results are shown in FIG. 6.

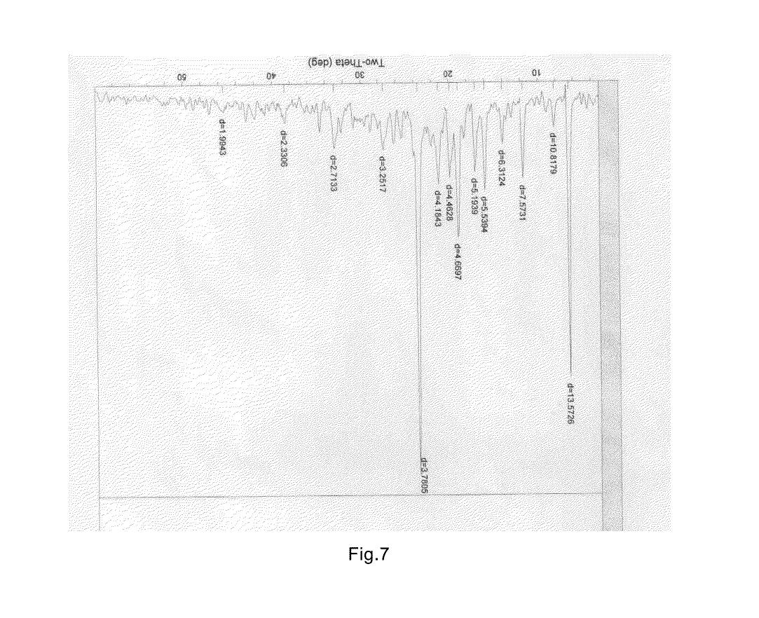

As measured by powder X-ray diffraction method, in range of diffraction angle 2θ (3-60°, the puerarin 0.5 hydrate (prepared by method of Example 5) had corresponding characteristic values at following 2θ values: about 6.5, 8.2, 11.7, 14.0, 16.0, 17.1, 19.0, 19.9, 21.2, 23.5, 27.4, 33.0, 38.6. The results are shown in FIG. 7.

### Example 25

### Stability Test

The sample of the puerarin hydrate above (prepared by method of Example 1) and anhydrous puerarin (anhydrous puerarin was prepared by: drying the hydrate as prepared in Example 1 at 90-105° C. in the presence of phosphorus pentoxide under vacuum for 24-72 hours or more to obtain an anhydrous puerarin, and the water content of anhydrous puerarin was usually lower than 1.0% as measured by Karl Fisher method) were sealed in penicillin bottles to perform accelerated stability test (chromatography conditions: chromatography column: C₁₈ (150 mm×4.6 mm, 5 μm); mobile phase: 0.1% citric acid solution-methanol (75:25); flow rate: 1 ml/min; temperature: room temperature; detection wavelength: 250 nm). It was surprisingly found that the puerarin hydrates of the present invention exhibited no significant changes in content, neither the related substance, while anhydrous puerarin gave a higher increase of related substance than the puerarin monohydrate when comparing 6 month results to 0 month results in the accelerated test (40° C., RH75%). Hygroscopicity test was performed according to the Pharmacopoeia of the People's Republic of China: about 5 g of anhydrous puerarin and the crystalline hydrate of the present invention were taken, placed in dry watch glasses with constant weight, weighed precisely. Samples were taken separately at 0 h and 48 h of the test at 25° C. and relative humidity of 75%, and hygroscopicity weight gain percentages were calculated. The results showed that the hygroscopicity of anhydrous puerarin was much higher than that of the hydrate of the present invention, which suggested that the crystalline puerarin hydrate of the present invention had better storage stability. The results are shown in Tables 1-4.

TABLE 1
Results of accelerated stability test of the puerarin
monohydrate of the present invention

| Sampling time | | Marked content | Related substance |
|---|---|---|---|
| (month) | Property | (%) | (%) |
| 0 | off-white powder | 99.5 | <2% |
| 1 | off-white powder | 99.3 | <2% |
| 2 | off-white powder | 99.6 | <2% |
| 3 | off-white powder | 99.4 | <2% |
| 6 | off-white powder | 99.3 | <2% |

TABLE 2
Results of long-term stability test of the puerarin
monohydrate of the present invention

| Sampling time | | Marked content | Related substance |
|---|---|---|---|
| (month) | Property | (%) | (%) |
| 0 | off-white powder | 99.5 | <2% |
| 3 | off-white powder | 99.6 | <2% |
| 6 | off-white powder | 99.5 | <2% |
| 9 | off-white powder | 99.4 | <2% |
| 12 | off-white powder | 99.4 | <2% |

TABLE 3
Results of accelerated stability test of the
puerarin 0.5 hydrate of the present invention

| Sampling time | | Marked content | Related substance |
|---|---|---|---|
| (month) | Property | (%) | (%) |
| 0 | off-white powder | 99.1 | <2% |
| 1 | off-white powder | 99.2 | <2% |
| 2 | off-white powder | 99.2 | <2% |
| 3 | off-white powder | 99.0 | <2% |
| 6 | off-white powder | 98.9 | <2% |

TABLE 4
Hygroscopicity test results

| | In comparison with |
|---|---|
| Sampling time (48 h) | 0 h, weight gain, % |
| puerarin monohydrate | 0.12 |
| puerarin 0.5 hydrate | 1.51 |
| Anhydrous puerarin | 4.26 |

### Example 26

### Effects on Arrhythmia Caused by Aconitine in Rats

20 SD rats, male and female, body weight 185±19 g, randomly divided into 2 groups, separately administrated via intravenous injection with physiological saline, puerarin monohydrate (prepared by method of Example 1) (which was added with cosolvent to form an injectable solution) 200 mg·kg⁻¹. The rats were anesthetized with 3% pentobarbital sodium 40 mg·kg⁻¹, and fixed in supine position on operating table. The rats were subjected to injection via sublingual vein, after 6 min, subjected to intravenous injection of 0.0025% aconitine at a constant rate, 2 μg·min⁻¹ (0.08 ml·min⁻¹), the time of ventricular premature beat (VP), ventricular tachycardia (VT), ventricular fibrillation (VF) and cardiac arrest (CA) were recorded, and corresponding dosages were calculated.

The results showed that the puerarin monohydrate group had a significantly increased dose of aconitine for causing VP, VT, VF and CA in rats, and there was significant difference between PW group and physiological saline group (see: Table 5).

TABLE 5
Effects on arrhythmia induced by aconitine in rats ( x̅ ± s, n = 10)

| | Dose | Dose of aconitine (μg · kg⁻¹) | | | |
|---|---|---|---|---|---|
| Drug | mg · kg⁻¹ | VP | VT | VF | CA |
| Physio- | 5 ml · kg⁻¹ | 26.3 ± 6.5 | 36.1 ± 6.8 | 52.2 ± 8.3 | 73.8 ± 7.9 |
| logical | | | | | |
| saline | | | | | |
| PW | 200 | 33.1 ± 7.3 | 44.9 ± 7.2 | 63.5 ± 7.7 | 95.1 ± 8.6 |

In comparison with PW group and physiological saline group (t test): p < 0.05.

### Example 27

### Effects of Puerarin Monohydrate on Inhibiting ACE Activity

24 Wistar rats, 280-300 g, were randomly divided into 3 groups, 8 rats per group, one group was control group, other two groups were separately 100 mg/kg, 200 mg/kg dose groups (which was added with cosolvent to form an injectable solution), subjected to intraperitoneal injection, the control group was administered with physiological saline. 60 min after administration, the rats were anesthetized with 1% pentobarbital sodium, subjected to thoracotomy and blood sample was taken from heart, the blood sample stood at room temperature for 30 min, centrifuged under 3500 r/min at low temperature for 10 min, the supernatant was taken and stored at about −65° C., for test.

Measurement of ACE activity: referring to document [WANG, Fumin, et al, Chinese Journal of Laboratory Medicine, 1983, 6(3):145-151], the results were subjected to statistical treatment: the results were expressed in x̅±S, and data were subjected to analysis of variance and t-test. The test showed that: as for rat blood serum ACE activity, when puerarin monohydrate group was compared with the control group, the rat blood serum ACE activity had a dose-dependent decrease (P<0.01), and puerarin monohydrate of different concentrations significantly inhibited ACE activity. The results are shown in Table 6.

TABLE 6
Effects of puerarin monohydrate on inhibiting ACE activity

| | | PLASMA | |
|---|---|---|---|
| | concentration | | inhibition |
| Group | (mg/kg) | ACE (IU) | rate (%) |
| Control group | 0 | 54.5 ± 6.48 | |
| puerarin monohydrate | 100 | 34.8 ± 6.19 | 36.1 |
| (prepared by method of | | | |
| Example 1) | | | |
| puerarin monohydrate | 200 | 26.7 ± 6.35 | 51.0 |
| (prepared by method of | | | |
| Example 3) | | | |

### Example 28

### Effects of Crystalline Puerarin Hydrate on Cerebral Ischemia in Rats

36 Wistar male rats, body weight 250-300 g, were randomly divided into 3 groups: sham operation group, ischemia-reperfusion group, and the present invention drug group, 12 rats per group. The rats of the present invention drug group were intragastrically administered with physiological saline suspension of puerarin monohydrate (prepared by method of Example 1) in a dose of 400 mg/kg, volume of 2 ml, twice per day, for consecutive 7 days, and cerebral ischemia operation was performed 60 min after the last administration; the rats of sham operation group and the ischemia-reperfusion group were administered separately with isometric physiological saline 2 ml.

The rats were anesthetized with 10% chloral hydrate (3.5 ml/kg) by intraperitoneal injection, fixed at supine position, and molded by a modified Zea Longa suture method referring to document (Journal of Shanghai Jiaotong University (medical Science), 2007, 27(10): 1218-1222); suture was inserted only 10 mm in sham operation group, and the other steps were the same as the model groups. Models were successfully made when the animals showed Homer syndrome and opposite side movement disorders after coming around. After successful modeling, the rats of the present invention drug group were administered twice per day in the same dose as above, while those of the sham operation group and the ischemia-reperfusion group were administered isometric physiological saline 2 ml. Sutures were drawn out 2 hours after modeling, the rats were executed 24 hours after reperfusion, and their brain weights were measured rapidly. 10% brain tissue homogenates were prepared at low temperature with physiological saline, centrifuged under 3000 r/min for 10 min at low temperature, the deposit was discarded, and the supernatant was taken and measured with commercial kits of SOD, MDA, NO, NOS according to their specifications to determine contents or activities of SOD, MDA, NO, NOS. The results are shown in Table 7.

TABLE 7
Effects of the present invention drug on NO, NOS, MDA,
SOD in brain tissues of rats after ischemia-reperfusion

| | | NOS | MDA | SOD |
|---|---|---|---|---|
| | NO (μmol/L) | (U/ml) | (nmol/ml) | (U/ml) |
| Sham operation | 21 ± 5.32 | 17 ± 3.54 | 1.63 ± 0.39 | 285 ± 21.49 |
| group | | | | |
| Ischemia- | 32 ± 8.71 | 28 ± 5.86 | 3.67 ± 0.66 | 216 ± 19.72 |
| reperfusion group | | | | |
| The present | 26 ± 6.13 | 23 ± 5.22 | 2.54 ± 0.57 | 243 ± 19.66 |
| invention drug | | | | |
| group | | | | |

The results showed that in comparison with the sham operation group, SOD activity in brain tissue of rats of the ischemia-reperfusion group decreased significantly, while MDA, NO contents and NOS activity increased significantly (P<0.01); in comparison with the ischemia-reperfusion group, SOD activity in brain tissue of rats of the present invention drug group increased significantly, NOS activity decreased, and MDA and NO contents significantly decreased (P<0.01).

Some researches show that NO is a messenger small molecule with biological activity, which can permeate cell membrane freely, and acts on target molecules in cells. In vivo, the generated NO is quickly inactivated by oxidization, and present in the form of nitrate and nitrite in external and internal liquids of cells. NO at low concentration can result in vasodilatation, inhibit platelet aggregation and adhesion, down regulate ionic channels as regulated by glutamic acid, prevent calcium overload in cells, and thus exhibit protection effects on cells; but NO at high concentration can react with superoxide anion to form superoxide nitrite ions, while superoxide nitrite ions can decompose to form free radicals of OH⁻ and NO²⁻, so that cell membrane was subjected to lipid peroxidation, resulting in intensive neurotoxicity at cell membrane level, and even resulting in death of neurons.

Some researches show that NO plays an important role in the pathogenesis of cerebral ischemic injury, and NO content increases several minutes after ischemia, then slowly decreases, and NO increases again during reperfusion. The mechanism thereof could be that: neurons are impaired after cerebral ischemia, the process of depolarization of cell membrane is enhanced, excitatory amino acids such as presynaptic glutamic acid increases significantly, so that extracellular glutamic acid concentration increases, which activates N-methyl-D-aspartic acid (NMDA) receptor, the activated NMDA receptor elevates postsynaptic Ca²⁺ internal flow, activates nitric oxide synthase NOS, promotes NO generation; secondly, after cerebral ischemia, ATP is consumed in large amount, which results in energy metabolism disorder and decrease in cAMP-dependent protein kinase activity, and since NOS dephosphrization increases NOS activity, the generation of NO is promoted.

Nitric oxide synthase is a rate-limiting enzyme NOS for nitric oxide synthesis. In early stage of cerebral ischemia, NO generated by NOS mediation improves blood supply in ischemic region via vasodilatation, which has short-term protection effect, but with the generation of NO in a large amount, neurotoxicity mediated by NOS in ischemic region is dominant and increases ischemic injury; in late stage, cerebral ischemic tissues are injured, inflammatory reaction stimulates macrophages, microglia, neuron can generate induced NOS in large amount, while induced NOS can slowly but persistently generate NO, and the generation and release of excessive NO aggravates neuron damage. Hence, NO release can be significantly reduced by reducing induced NOS, so as to reduce cytotoxic effect of NO and protect brain cells.

The test results of this example show that after cerebral ischemia-reperfusion in rats, the contents of NO and NOS in brain tissue increase significantly, which indicates NO and NOS are involved in pathogenesis of cerebral ischemia-reperfusion injury; after treatment with the present invention drug, the contents of NO and NOS decrease significantly, which indicates that the present invention drug has protection effect on cerebral ischemia-reperfusion injury by regulating NOS activity and NO content.

Cerebral ischemia-reperfusion injury can result in membrane lipid peroxidation of brain cells, generate excessive oxygen free radicals, injure brain cell membrane. During cerebral ischemia, the deficiency of oxygen and energy metabolism substance in tissue results in reduction of ATP, failure of ionic pump, activity decrease of Na⁺—K⁺-ATP enzyme, so that a large amount of Na⁺ flows internally, K⁺ flows externally, Cl⁻ and H₂O enter cells passively, resulting in acute osmotic swelling and death of nerve cells. Na⁺ internal flow and K⁺ external flow lead to decrease of cell membrane potential and depolarization, voltage-dependent Ca²⁺ channel opening, and large Ca²⁺ internal flow; in the meantime, due to the release of K⁺, protein kinase C and transmitters, receptor-dependent Ca²⁺ channel opens, Ca²⁺ internally flows in large amount. Intracellular calcium overload can result in an increase of oxygen free radicals, an enhancement in arachidonic acid metabolism, increase of excitatory amino acids transmitter release, while excitatory amino acids can further induce intracellular Ca²⁺ overload which result in apoptosis of nerve cells. One of important facts for ischemia-reperfusion is the increase of oxygen free radicals.

Various oxygen free radicals attack unsaturated fatty acids in biomembranes, induce lipid peroxidation, form a series of lipid free radicals and degradation product MDA. MDA as a metabolism product of lipid peroxidation between oxygen free radicals and unsaturated fatty acids in biomembranes can indirectly indicate the change of content of oxygen free radicals.

SOD is a main enzymatic defensive system against oxygen free radicals in cells, which scavenges superoxide anions via disproportionation, protects organism from being attacked by free radicals. The level of SOD activity indirectly reflects the ability of scavenging oxygen free radicals in organism.

Hence, the present invention drug can significantly reduce lipid peroxide MDA and NO content in ischemic brain tissue, elevate SOD content in brain tissue of rats, enhance the ability of scavenging free radicals, reduce NOS activity, and alleviate damage on brain tissue caused by reaction of free radicals. It can be seen that the present invention drug can inhibit various routes for generating free radicals, protect cell membrane structure, and facilitate reducing cerebral vascular permeability and improving hemorheological indexes. The improvement of brain microcirculation can resist the damage caused by oxygen free radicals after cerebral ischemia-reperfusion and thus protect brain. It can be used for the treatment or prophylaxis of ischemic encephalopathy, cerebral infarction, cerebral thrombosis and sequels thereof, for the improvement of brain microcirculation, for potential prophylaxis and treatment of cataract, and for manufacturing a medicament or health care product for treatment and prophylaxis of corresponding diseases.

### Example 29

### Effects of Crystalline Puerarin Hydrate on Chronic Ocular Hypertension of Rabbits

Chronic Ocular Hypertension Models:

32 rabbits, half male and half female, were subjected to sub-conjunctive injection with 0.5 mg of dexamethasone under corneal limbus, once every other day, for consecutive 3 weeks, to obtain chronic ocular hypertension models, then randomly divided into physiological saline group, betaxolol group [0.5% (W/V)], puerarin hydrate group [1% (W/V)]. The maximum decrease of intraocular pressure was measured within 24 hours. The results are shown in Table 6.

TABLE 8
Effects crystalline puerarin hydrate on
chronic ocular hypertension of rabbits

| | Maximum decrease |
|---|---|
| | of intraocular |
| | pressure (mmHg) |
| Physiological saline | 0.9 |
| Betaxolol | 1.9 |
| Sample 1 as prepared in Example 1 | 2.6 |
| Sample 2 as prepared in Example 2 | 2.4 |
| Sample 5 as prepared in Example 5 | 2.5 |
| (puerarin 0.5 hydrate) | |

Although the embodiments of the present invention are illustrated in details, those skilled in the art would understand that according to the teachings as disclosed, these details can be modified and changed, and all these changes are within the protection scope of the present invention. The whole protection scope of the present invention is defined by the appended claims.

What is claimed is:

1. A puerarin hydrate, of which the molecular formula is C₂₁H₂₀O₉.n H₂O, wherein n is a value selected from 0.4-1.3 or 0.8-1.3.

2. The puerarin hydrate according to claim 1, wherein n is 0.5, 0.8, 0.85, 1, 1.05, 1.2, 1.25 or 1.3.

3. A method for preparing the puerarin hydrate of claim 1 or 2, which is any one of the following methods A to E:
Method A:
In a reaction container, subjecting Radix Puerariae powder to cold extraction, ultrasonic extraction, microwave extraction or refluxing extraction one or more times with one or more of water or C1-C6 lower alcohols, combining, filtering, filtering with microporous membrane or ceramic membrane, concentrating filtrate, concentrating to obtain Radix Puerariae extractum, adding water, adjusting pH to 3-5 with an acid, then adjusting pH to 6-9 with one or more of a base or sodium carbonate or sodium hydrogen carbonate, filtering, then extracting with one or more solvents of C3-C6 lower alcohols or C3-C8 lower ketones, such as hexone, or C2-C8 lower esters, drying by evaporation to obtain a solid, adding water, then extracting with one or more solvents of C3-C6 lower alcohols or C3-C8 lower ketones, such as hexone, or C2-C8 lower esters, concentrating, standing, filtering, performing recrystallization one or more times with one or more solvents of water, C1-C6 lower halogenated hydrocarbons, C3-C8 lower ketones, such as acetone, C2-C8 lower esters, C1-C6 lower alcohols, such as methanol, ethanol, isopropanol, C1-C6 lower fatty acids, tetrahydrofuran, acetonitrile, filtering, washing with water, and drying the resultant solid to obtain the crystalline puerarin hydrate;
Method B:
To Radix Puerariae extractum or Radix Puerariae total flavonoids or Radix Puerariae water extract concentrate or Radix Puerariae alcohol extract concentrate, adding one or more of water or C1-C6 lower alcohols, filtering with microporous filtration membrane (including ceramic membrane), passing through a neutral alumina chromatography column, silica gel chromatography column and macroporous resin or polyamide chromatography column or glucose gel resin chromatography column, eluting with one or more of water or C1-C6 lower alcohols, C1-C6 lower halogenated hydrocarbons, concentrating or concentrating with ultrafiltration membrane, standing, filtering, performing recrystallization one or more times with one or more crystallization solvents of water, C1-C6 lower halogenated hydrocarbons, C3-C8 lower ketones, such as acetone, C2-C8 lower esters, C1-C6 lower alcohols such as methanol, ethanol, isopropanol, C1-C6 of lower fatty acids, tetrahydrofuran, acetonitrile, filtering, washing with water, drying the resultant solid to obtain the crystalline puerarin hydrate;
Method C:
To Radix Puerariae extractum or Radix Puerariae total flavonoids or Radix Puerariae water extract concentrate or Radix Puerariae alcohol-water extract concentrate, adding one or more of water or C1-C6 lower alcohols, subjecting to filtration or concentration one or more times with one or more of microporous filtration membrane, hollow fiber membrane, ceramic membrane, ultrafiltration membrane, nanofiltration membrane, performing recrystallization one or more times with a crystallization solvent of one or more of water, C1-C6 lower halogenated hydrocarbons, C3-C8 lower ketones, such as acetone, C2-C8 lower esters, C1-C6 lower alcohols such as methanol, ethanol, isopropanol, C1-C6 of lower fatty acids, tetrahydrofuran, acetonitrile, filtering, washing with water, drying the resultant solid to obtain the crystalline puerarin hydrate;
Method D:
To Radix Puerariae extractum or Radix Puerariae total flavonoids or Radix Puerariae water extract concentrate or Radix Puerariae alcohol-water extract concentrate, subjecting to filtration or concentration one or more times with one or more of hollow fiber membrane, ceramic membrane, ultrafiltration membrane, nanofiltration membrane, performing recrystallization one or more times in a magnetic field with a magnetic flux of 0.1-5 Tesla with a crystallization solvent of one or more of water, chloroform, C3-C6 lower ketones, such as acetone, C2-C8 lower esters, C1-C6 lower alcohols such as methanol, ethanol, isopropanol, C1-C6 of lower fatty acids, tetrahydrofuran, acetonitrile, filtering, washing with water, drying to obtain the crystalline puerarin hydrate; and
Method E:
Subjecting an anhydrous puerarin to hydration or recrystallization.

4. A pharmaceutical composition comprising the puerarin hydrate of claim 1 or 2.

5. The pharmaceutical composition according to claim 4, which is a freeze-dried powder injection, a small volume injection, a large volume infusion solution, a tablet, a capsule, a granule, an eye drops, or an ophthalmic gel.

6. The pharmaceutical composition according to claim 4, which further comprises a cyclodextrin or a cyclodextrin derivative, wherein the mass ratio or weight ratio of the crystalline puerarin hydrate to the cyclodextrin or cyclodextrin derivative is 1:5 to 1:60, preferably, the cyclodextrin or cyclodextrin derivative is one or more selected from the group consisting of 2-hydroxypropyl-β-cyclodextrin, 3-hydroxypropyl-β-cyclodextrin, and sulfobutyl ether-β-cyclodextrin (SBE-β-CD).

7. Use of the puerarin hydrate of claim 1 or 2 in the manufacture of a medicament for the treatment or prophylaxis of any one of the following diseases:
hypertension, coronary heart disease, pulmonary heart disease, heart failure, angina pectoris, myocardial infarction, cardiogenic shock, arrhythmia, myocarditis, ischemic encephalopathy, cerebral infarction, cerebral thrombosis and sequela thereof, improvement of brain circulation, vertebrobasilar ischemia dizziness, lower limb deep vein postthrombotic syndrome, diabetes, diabetic complications such as diabetic nephropathy, diabetic peripheral neuropathy, retinopathy, retinal arterial obstruction, vein occlusions, sudden deafness, ocular hypertension, glaucoma, or dyslipidemia.

8. Use of the puerarin hydrate of claim 1 or 2 in the manufacture of an angiotensin converting enzyme inhibitor.

9. Use of the puerarin hydrate of claim 1 or 2 in the manufacture of β receptor blocking agent.

10. A method for the treatment or prophylaxis of any one of the following diseases, comprising a step of administering an appropriate amount of the puerarin hydrate of claim 1 or 2, or the pharmaceutical composition of any one of claims 4-6:
hypertension, coronary heart disease, pulmonary heart disease, heart failure, angina pectoris, myocardial infarction, cardiogenic shock, arrhythmia, myocarditis, ischemic encephalopathy, cerebral infarction, cerebral thrombosis and sequela thereof, improvement of brain circulation, vertebrobasilar ischemia dizziness, lower limb deep vein postthrombotic syndrome, diabetes, diabetic complications such as diabetic nephropathy, diabetic peripheral neuropathy, retinopathy, retinal arterial obstruction, vein occlusions, sudden deafness, ocular hypertension, glaucoma, or dyslipidemia.
